(19) United States
(12) Plant Patent Application Publication
(10) Pub. No.: US 2022/0095518 P1
(43) Pub. Date: Mar. 24, 2022

(54) Clematis Plant Named 'Evipo108'

(50) ;

(71) Applicants: Mogens Nyegaard Olesen, Fredensborg (DK); Raymond J. Evison, Vale (GB)

(72) Inventors: Mogens Nyegaard Olesen, Fredensborg (DK); Raymond J. Evison, Vale (GB)

(21) Appl. No.: 17/300,635

(22) Filed: Sep. 7, 2021

(30) Foreign Application Priority Data
Sep. 21, 2020 (EU) 2020/2280

(51) Int. Cl. A01H 6/72

(52) U.S. Cl. PLT/228

(57) ABSTRACT

A new Clematis plant with a compact growth habit, profuse, white flowers, and continuous summer flowering. The variety successfully propagates from softwood cuttings and is suitable for cultivation in commercial nursery culture. This new and distinct variety has shown to be uniform and stable in the resulting generations from asexual propagation from vegetative cuttings.

Botanical classification: Genus: Clematis Species: viticella.

Variety denomination: ‘Evipo108’.

This application claims priority to Plant Breeder's Rights Application Number 2020/2280, which was filed at the Community Plant Variety Rights Office in the European Union on Sep. 21, 2020, the contents of which are hereby incorporated by reference for all purposes.

### SUMMARY OF THE CLAIMED PLANT

The present invention constitutes a new and distinct variety of Clematis plant which originated from a controlled crossing between the female seed parent, an un-named seedling, and the male pollen parent, an un-named seedling. Both parent varieties are non-patented.

The two parents were crossed during the summer of 2007 and the resulting seeds were planted the following winter in a controlled environment in Guernsey, Channel Islands, United Kingdom. The new variety named ‘Evipo108’ originated as a single seedling from the stated cross.

The new Clematis plant may be distinguished from its female seed parent and male pollen parent by the following characteristics. The female seed parent has a growth height of 75 cm while the new variety grows to 30 cm. The male seed parent grows to a height of 75 cm while the new variety grows to 30 cm.

The objective of the hybridization of this Clematis plant was to create a new and distinct variety for nursery culture with unique qualities such as:
1. Uniform and abundant white flowers;
2. Ultra-compact growth, making the variety suitable for a variety of container culture uses; and
3. Improved disease resistance.

This combination of qualities was lacking in Clematis plants that were in commercial cultivation and the qualities have been substantially achieved in the new variety.

‘Evipo108’ was selected by Mogens N. Olesen and Raymond J. Evison in their Clematis development program in the Channel Islands, United Kingdom in 2008. Asexual reproduction of ‘Evipo108’ by means of vegetative cuttings and traditional layering was first performed by Mogens N. Olesen and Raymond J. Evison in the nursery during the summer of 2008. This initial and subsequent asexual propagations have demonstrated that the characteristics of ‘Evipo108’ are true to type and are transmitted from one generation to the next.

### BRIEF DESCRIPTION OF THE DRAWING

The accompanying color illustrations show as true as is reasonably possible to obtain in color photographs of this type the typical characteristics of the buds, flowers, leaves, and stems, of ‘Evipo108’.

Specifically illustrated in FIG. 1 of the drawings shows a cluster of open flowers on a branch, detached tepals revealing reproductive flower parts, and the underside of an open flower.

FIG. 2 shows the under and upper surface of leaflets.

FIG. 3 illustrates the upper surface of an open flower, leaves attached to the branch showing juvenile and mature wood.

FIG. 4 shows a close up image of an open flower. These plants illustrated are 2 years of age.

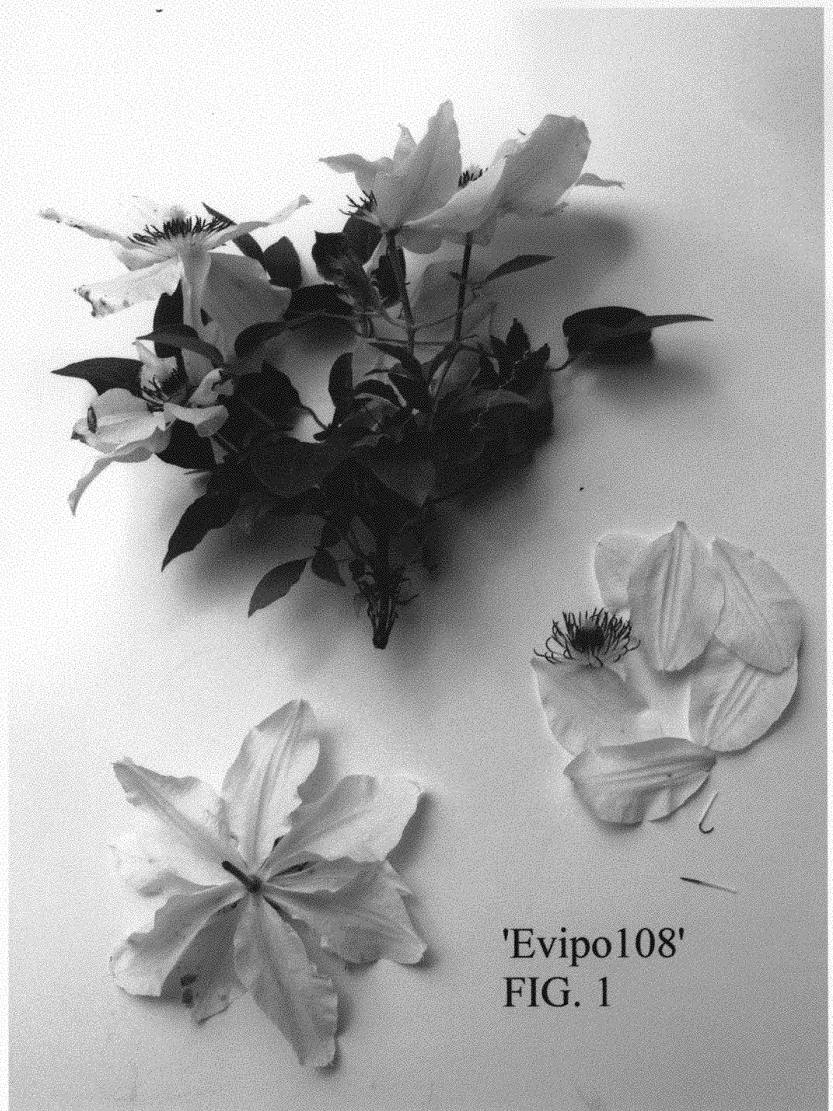

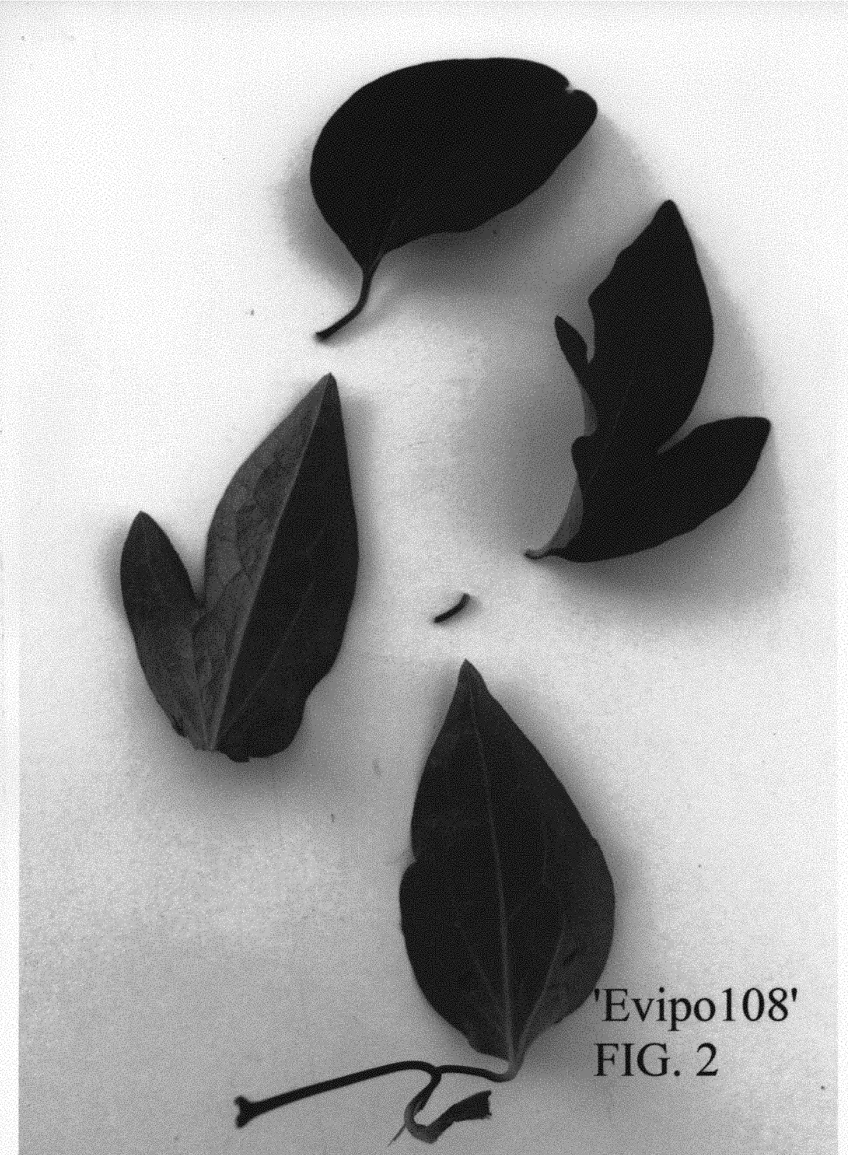

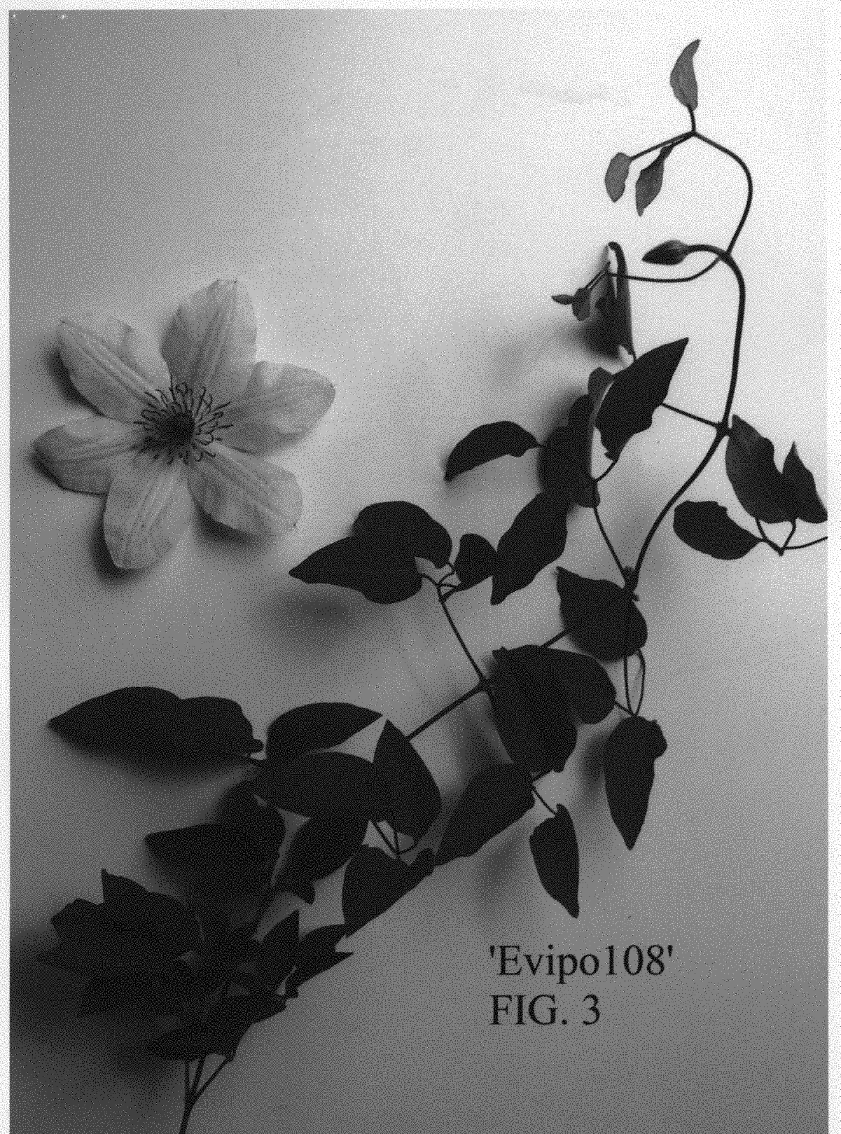

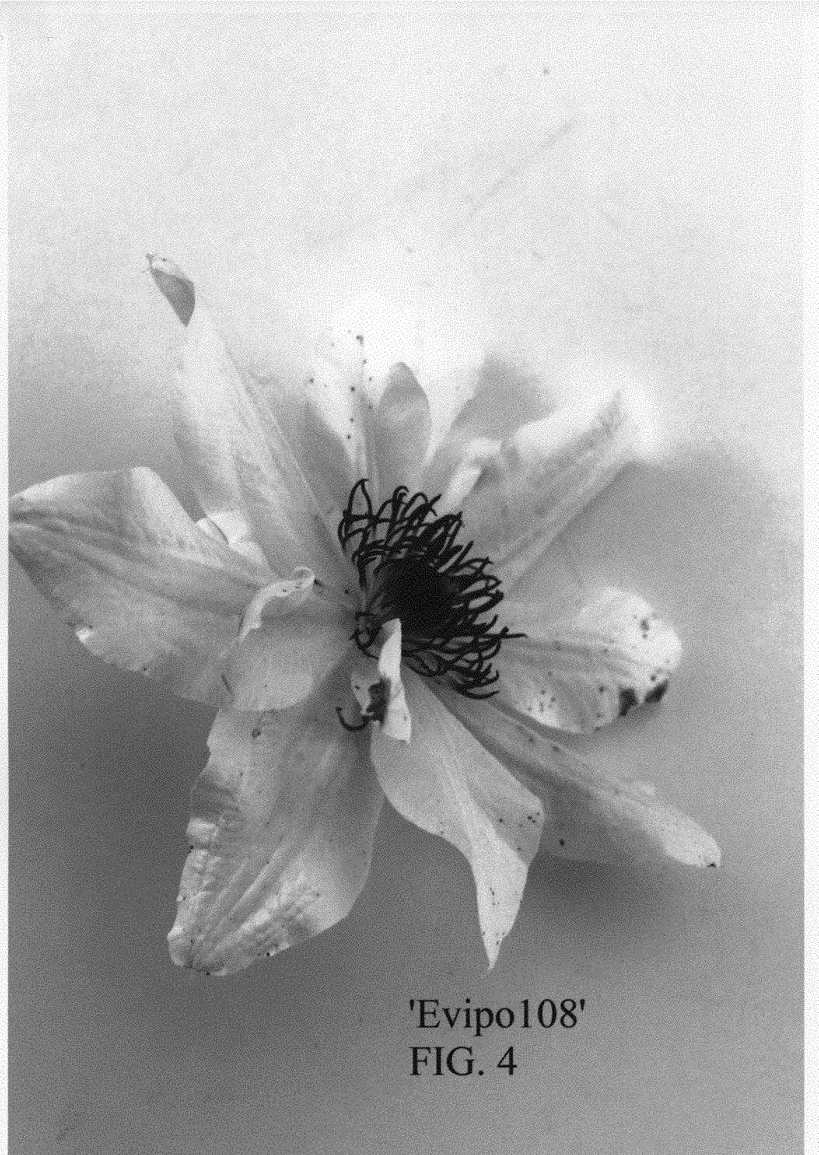

### DETAILED DESCRIPTION OF THE VARIETY

The following is a detailed description of ‘Evipo108’, as observed in its growth outdoor throughout the flowering period in Odense Denmark. Observed plants were cultivated for a period of 24 months in 2 liter containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used. For a comparison, several physical characteristics of the Clematis variety ‘Evipo093’ described and illustrated in U.S. Plant Pat. No. 31,611 are compared to ‘Evipo108’ in Chart 1.

CHART 1

| | ‘Evipo108’ | ‘Evipo093’ |
|---|---|---|
| Flower diameter | 140 mm | 90-130 mm |
| Tepal upper | White Group 155C | White Group 155B |
| surface | with light intonations | |
| | of Greyed-Red Group | |
| | 182D | |
| Tepal quantity | 6 tepals, with 4 to 5 | 6 |
| | tepaloids | |

Flower and flower bud:
   Blooming habit.—Continuous. The natural flowering period is generally from April to September.
   Flower bud.—Size: Normally 18 to 22 mm in length. Bud diameter is 6 mm. Bud form: Long campanulate, broad based. Bud color: Yellow-Green Group 144A. Texture: Pubescent.
   Pedicel.—Surface Texture: Pubescent. Length: On average 40 to 60 mm in length with 2 mm diameter. Color: Yellow-Green Group 144A. Strength: Moderately strong.
   Receptacle.—Surface Texture: Lightly pubescent. Shape: Broad funnel. Size: 1.5 mm (h)×6 mm (w). Color: Yellow-Green Group 144A.
   Flower arrangement.—Location on vine: New growth only. Borne: Clusters of 3 to 9 flowers on a flowering branch.
   Flower bloom.—Size: Average 140 mm diameter and 40 mm in depth. Profile: Open flowers are flat. Fragrance: None. Lasting Quality: Flowers normally remain up to 14 days on the plant.
   Tepals.—Tepal Color: Upon opening, the upper surface is White Group 155C with light intonations of Greyed-Red Group 182D. The lower surface is White Group 155 C with a central bar of Yellow-Green Group 144D. Quantity: Normally 6 tepals, with 4 to 5 tepaloids. Tepaloids are 35×10 wide with twisted margins. Size: 65 mm in length by 30 mm wide. Texture: Smooth with three ridges running length of central bar. Shape: Individual tepal shape is broadened elliptical. The tepal apex is obtuse with small point at center. The tepal base is acute. Apex Recurvature: Strong . Tepal Cross section: Reflexed. Margins: Entire. Moderate to strong undulations of margin observed. Persistence: Tepals will drop easily after flowers are fully matured.
   Reproductive organs.—Pollen: None observed. Anthers: Size: 6 mm in length. Color: Greyed-Purple Group N186A. Quantity: On average, 70. Filaments: Color: Green White Group 157A. Length: 6 to 10 mm. Pistils: Quantity: On average, 25. Stigmas: Superior in location relative to the length of the filaments and the height of the anthers. Styles: Color: Green-White Group 157D. Length: 11 mm.
   Seed head characteristics.—Seed not observed to date.
Plant:
   Plant form.—Very compact, mounding.
   Plant growth.—
   Size.—Seasons growth attains 30 cm in height. Average spread is 50 cm.
   Stems.—Color: Juvenile stems are Yellow-Green Group 144A with intonations of Greyed-Orange Group 164A. Mature stems are Greyed-Purple Group 183B. Internodes: On average, cm between nodes. Length: Normally 10 cm from the base of the plant to the flowering portion of the stem. Diameter: About 3 to 4 mm. Texture: Mature stems are ribbed.
   Plant foliage.—Leaf characteristics: Deciduous. Arrangement: Trifoliate. Leaf Size: Compound leaves are about 90 mm (l)×110 mm (w). Leaflets are about 42 mm (l)×32 mm (w). Abundance: On average leaves per 10 cm of stem. Leaf Color: Juvenile upper Yellow Group 144A. Juvenile lower Yellow Group 144A. Mature upper Green Group 137A. Mature lower Yellow-Green Group 146B. Stipules: Absent. Petioles: Size: Normally 40 mm in length by 2 mm diameter. Texture: Smooth. Color: Yellow-Green Group 144A with intonations of Greyed-Purple 183A. Petioloule: Size: About 10 mm in length by 1.5 mm diameter. Texture: Smooth. Color: Yellow-Green Group 144A with intonations of Greyed-Purple 183A. Leaflet Shape: Ovate. The base is rounded or oblique, apex acute. Margin: Entire. Occasionally a deep cleft in the margin. Surface: The upper side is smooth, the lower side is smooth. Thickness: Moderate. Glossiness: Moderately glossy.
   Disease resistance.—Subject to any disease that normally attacks the species. However the variety is more tolerant to Clematis wilt, Ascochyta clematidina, than some Clematis known to the inventors.
   Cold hardiness.—The variety is tolerant to USDA Hardiness Zone 6.
   Heat tolerance.—The variety has been found to be suitable for climate conditions found in the American Horticulture Society heat zone 7.

We claim:

1. A new and distinct variety of Clematis plant named ‘Evipo108’, substantially as described and illustrated , due to its abundant white flowers with good keepability, attractive long lasting foliage and compact growth, year round flowering under glasshouse conditions, suitability for production from softwood cuttings in pots, durable flowers and foliage which make the variety suitable for distribution in the floral industry.
